(19) United States
(12) Patent Application Publication
(10) Pub. No.: US 2006/0104922 A1
(43) Pub. Date: May 18, 2006

(54) Tooth whitening compositions and methods for using the same

(76) Inventors: J. Gail Tarver, Lehi, UT (US); Ben D. Card, Mapleton, UT (US); Michael R. LaDow, Provo, UT (US); Donald H. Livingstone, Provot, UT (US)

Correspondence Address: Berne S. Broadbent;KIRTON & McCONKIE, 60 East South Temple Street, P.O. Box 45120, Salt Lake City, UT, 84145-0120, US

(21) Appl. No.: 11/217,275

(22) Filed: Sep. 1, 2005

Related U.S. Application Data
(63) Continuation-in-part of application No. 10/442,559, filed on May 21, 2003.

(51) Int. Cl. A61K 8/49; A61K 8/22

(52) U.S. Cl. 424/53

(57) ABSTRACT

A tooth whitening system that effectively conceals tooth discoloration within a short period of time. The system includes a single, spectrally pure, substantially violet dye and a carrier for applying the dye to teeth.

### RELATED APPLICATIONS

This application is a continuation-in-part of U.S. patent application Ser. No. 10/442,559, filed May 21, 2003, entitled, “TOOTH WHITENING COMPOSITIONS AND METHODS FOR USING THE SAME,” incorporated herein in its entirety by reference.

### BACKGROUND

1. Field of the Invention

The present invention relates to compositions for whitening teeth. More particularly, the present invention relates to a dye suitable for application to teeth that causes the teeth to appear whiter, as well as a method of applying that composition.

2. Background

Tooth whitening is increasingly recognized as a relatively quick and inexpensive way to improve one's appearance. Accordingly, many compositions and methods for obtaining whiter teeth are presently known.

Many such compositions and methods, however, require professional application and are thus both costly and time consuming. Indeed, all laser and some bleaching procedures must be performed by dentists in a dental office, and can cost thousands of dollars, depending on the number of teeth to be treated and the severity of their discoloration. Even some compositions capable of superficial application require the supervision of a dental professional due to the potency of the substances used and the risks inherent in their application.

Most presently known topically applied compositions for whitening teeth rely on invasive chemical components to break down pigments internally disposed in teeth. Even when properly applied, these chemical components have been known to cause increased sensitivity in both teeth and gingival tissues in some people, and moderate to severe pain in others. One theory is that increased sensitivity and pain result from the bleaching agent working its way through pores that naturally occur in teeth and thus into the pulp chamber where nerves are located; the bleaching agent may also directly cause sensitivity to gingival tissue that it comes into contact with. Some have also posited that certain carriers of the chemicals used to whiten teeth can cause teeth to become somewhat desiccated or dehydrated, causing increased internal fluid pressure and pain within the pulp chamber.

Because of the high costs and the risks associated with traditional bleaching compositions and techniques, less potent compositions that are available for home use have become increasingly popular and are widely available as over-the-counter products. Although such compositions avoid the disadvantages of more potent bleaching systems, such over-the-counter compositions intended for home use are typically less effective in removing or concealing tooth discoloration. They also typically require increased frequency and duration of use compared to professionally applied compounds.

Accordingly, what is needed is an effective, non-invasive, non-destructive tooth whitening system that does not have the high costs or risk of painful effects inherent in much of the prior art. Such compositions and methods for whitening teeth are disclosed and claimed herein.

### SUMMARY OF THE INVENTION

The present invention is a tooth whitening system that effectively conceals tooth discoloration within a short period of time. According to certain embodiments, a tooth whitening composition in accordance with the present invention comprises a food product capable of being deposited onto a person's teeth during ordinary consumption. In this manner, the tooth whitening composition of the present invention minimizes the time and effort required for tooth whitening while maximizing whitening results.

An object of the present invention is to provide a tooth whitening system that incorporates a dye having a dominant peak absorption wavelength to effectively mask tooth discoloration.

Another object of the present invention is to provide a topically applied tooth whitening system that provides maximum whitening effect without causing tooth sensitivity.

Another object of the present invention is to provide an inexpensive tooth whitening system that is safe for home use.

It is a further object of the present invention to provide a tooth whitening system capable of producing effective results within a short period of time after its initial application.

These and other features and advantages of the present invention will be set forth or will become more fully apparent in the description that follows. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 is a comparative chart of ranges of wavelengths absorbed and the corresponding colors that are absorbed and reflected (visually apparent) by various dyes;

FIG. 2 is a spectral analysis showing a comparison between the absorption spectra of a dyes having peak absorption wavelengths in the visible range of red and blue, compared to a dye having a dominant peak absorption wavelength in the visible range of violet;

FIG. 3 is a spectral analysis of one embodiment of a dye in accordance with the present invention;

FIG. 4 is a spectral analysis of a second embodiment of a dye in accordance with the present invention;

FIG. 5 is a spectral analysis of a third embodiment of a dye in accordance with the present invention; and

FIG. 6 is a spectral analysis of a fourth embodiment of a dye in accordance with the present invention.

### DETAILED DESCRIPTION OF THE INVENTION

The present invention may be embodied in various forms without departing from its spirit or essential characteristics. The embodiments described herein are thus to be considered in all respects only as illustrative and not restrictive. The scope of the invention is, therefore, indicated by the appended claims rather than by the following descriptions. All changes that come within the meaning and range of equivalency of the claims are to be embraced within their scope.

As used in this specification, the term “dye” refers to any FD&C dye, D&C dye, FD&C lake, acid dye, or natural food color such as carmine, carotene, annatto, turmeric, curcumin, blueberry, or other natural food colors known in the art, whether used individually or in combination. The term “food product” refers to any nutriment in solid or liquid form that is safe either for human consumption or for application to the mucous membranes of the mouth (including mouthwashes, dentifrices, and the like).

As used in this specification, “visible spectrum” and “light” refers to the band of electromagnetic radiation ranging from wavelengths of approximately 400 to approximately 700 nm, corresponding to the sensitivity of the human eye.

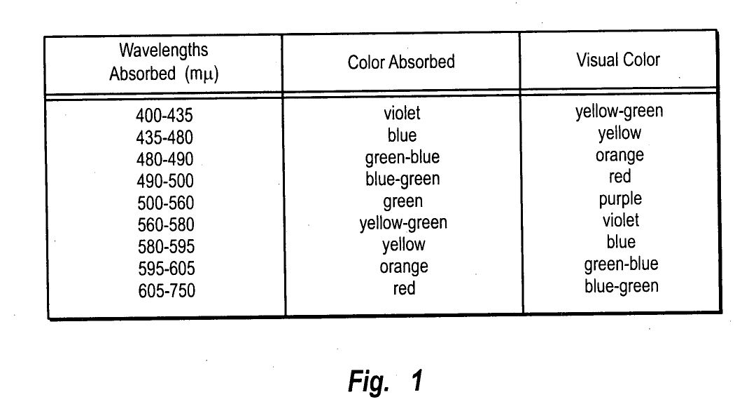

The human eye has three types of cone cells that have peak responses to light with wavelengths that correspond to red, green, and blue-violet (the wavelengths corresponding to these colors being set forth in FIG. 1). “White light” refers to any electromagnetic radiation that stimulates all three types of cone cells in the human eye—red, green, and blue-violet—in substantially the same proportions as do the emissions from a body at a white heat. We can designate the stimulation of the three types of cone cells by white light as R:G:B, having a ratio of 1:1:1. “Color” indicates light that is not perceived as white because it stimulates cone cells in a ratio R₁:G₁:B₁ that is not equal to 1:1:1. Each color R₁:G₁:B₁ has a “complementary color,” which we designate as R_{C}:G_{C}:B_{C}, such that white light is perceived when a color and its corresponding complementary color enter the eye simultaneously. This is because adding a color and its complementary color yields a ratio of 1:1:1, equal to the stimulations of the eye created by white light. In other words, the ratio of the stimulation to the eye provided by the combination of a color and its complementary color, expressed as R₁+R_{C}:G₁+G_{C}:B₁+B_{C}, is equal to 1:1:1.

As an example, light having a wavelength that is perceived as blue-violet stimulates cone cells receptive to that wavelength (blue-violet), and is represented in this model by a ratio of 0:0:1. Light having a wavelength that is perceived as yellow stimulates cone cells receptive to corresponding wavelengths (red and green), and is represented in this model by a ratio of 1:1:0. When these two colors are perceived simultaneously, the additive ratio of their components is 1:1:1. Accordingly, white light is perceived. Blue-violet and yellow are thus complementary colors of light.

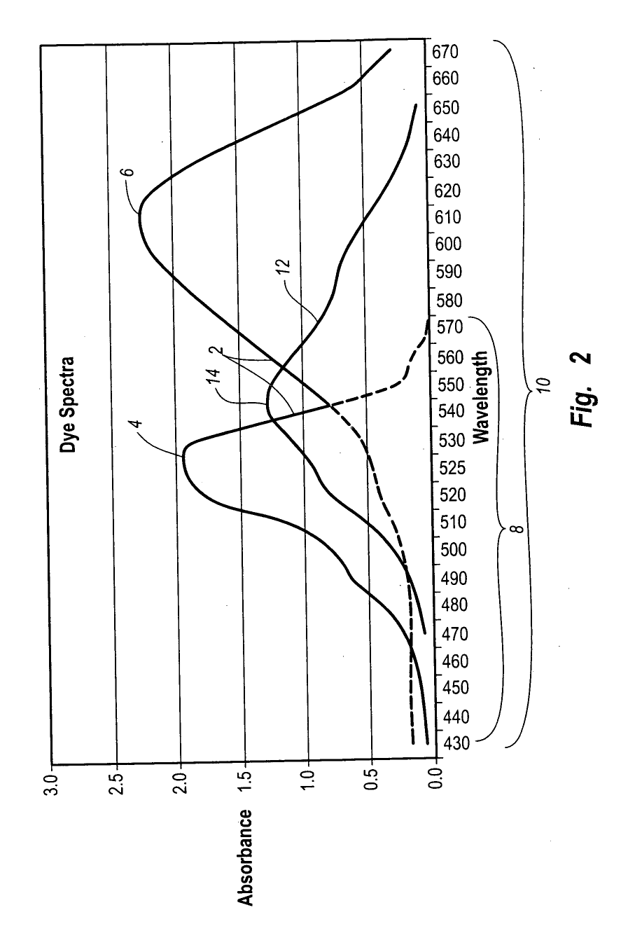

Conversely, a dye causes color perception by absorbing light having a particular wavelength. When a dye is illuminated by white light, one color is reflected (transmitted) by the dye; the complementary color of that color is absorbed by the dye. A spectral analysis of a dye can be used to illustrate, as in FIGS. 2 through 6, the wavelengths of light that the dye absorbs or transmits when illuminated by white light. The term “peak absorption wavelength” refers to that portion of the visible spectrum having a significantly higher level of absorption relative to its immediately adjacent portions.

Where all wavelengths of light corresponding to the visible spectrum are reflected from an object, the color of that object is perceived as white. Conversely, where all wavelengths of light corresponding to the visible spectrum are completely absorbed, the color of the object is perceived as black. When various wavelengths of light are absorbed differently, various colors are perceived.

If a white object is coated with a dye and then illuminated by white light, the light that is not absorbed by the dye is transmitted and perceived as color. The definition of a color thus perceived may be described either by referring to the wavelengths of the visible spectrum that the dye absorbed or by referring to the wavelengths of the visible spectrum that the dye transmitted. For the purposes of the present invention, all wavelengths used to numerically describe a dye are defined according to the wavelengths of the visible spectrum that the dye absorbs. For example, a dye that is perceived as red (e.g. it transmits wavelengths of the visible spectrum that the eye perceives as red), will be referred to herein as a dye that absorbs cyan-blue—the complimentary color of red when combining colors of light. A dye may also be described by those skilled in the art based upon the wavelengths of the visible spectrum that the dye transmits.

An exemplary non-invasive tooth-whitening composition suitable for home use is disclosed in U.S. Pat. No. 6,030,222 to Tarver (“Tarver”). Tarver teaches a whitening composition that includes a dye that, when absorbed by a tooth, causes the tooth to reflect a color of light that is whiter than the initial color of light reflected by the tooth. According to Tarver, the dye that most effectively causes this change in the light reflected by a tooth is a dye that reflects light having a wavelength that corresponds to a range from violet to blue-violet, this range of colors being a complementary color to the normal yellowish or off-white coloration of discolored teeth.

The effectiveness of the compositions disclosed in Tarver, however, is limited by the fact that the disclosed violet or blue-violet dye comprises unspecified quantities of red and blue dyes. The violet or blue-violet dye disclosed by Tarver has multiple peak absorption wavelengths, as illustrated at points 4 and 6 in FIG. 2. These multiple peak absorption wavelengths combine in the invention disclosed by Tarver to create a dye having a composite reflection or appearance in a range from violet to blue-violet. While theoretically a valid technique, such a combination of red and blue dyes is not as effective as the present invention. It appears that such combinations are subject to separation, either at a macroscopic or molecular level, once applied to teeth. Each component of the dye mixture may be absorbed differently by tooth surfaces, altering the light absorption characteristics of the initial dye mixture by leaving one of the peak absorption wavelengths more prominent in the dye after application to a tooth.

The compositions disclosed in Tarver present peripheral problems as well. The heterogeneous dye mixture taught by Tarver may cause discoloration of surrounding oral tissues because components of the dye mixture are preferentially absorbed by different oral tissues. When these dye mixtures are applied to teeth, the gums, tongue and surrounding oral tissues may display a red tint due to the continued presence of red dye within the initial dye mixture.

The present invention relies upon a single dye or combination of dyes having a dominant peak absorption wavelength corresponding to the complementary color of violet or blue-violet light, rather than, as Tarver discloses, a dye or combination of dyes having multiple peak absorption wavelengths that, taken together, are perceived as corresponding to the complementary color of violet or blue-violet. The whitening effect of a dye having a dominant peak absorption wavelength is significantly superior to a compound comprising multiple peak absorption wavelengths, as disclosed in Tarver. Further, a compound according to the present invention has been demonstrated to cause significantly less discoloration of adjacent oral tissues compared to the compound disclosed in Tarver.

Referring to FIG. 1, it is apparent that when an object is illuminated by white light, the wavelength of light corresponding to the color that is perceived—i.e. the light reflected from the object—is the complementary color of the wavelength of light corresponding to the color absorbed by the object. For example, a color perceived as blue-violet is caused by absorption of all yellow light (i.e., light having a wavelength between 560-595 nm). Likewise, a color perceived as yellow is caused by absorption of all blue-violet light (i.e. light having a wavelength between 400-480 nm). Yellow and blue-violet are complementary colors of light.

The present invention exploits the relationship between colors by disclosing a dye having a dominant peak absorption wavelength that is a complementary color to the absorption wavelengths typical of a yellowed tooth. When such a dye is added to the surface of the yellowed tooth, all of the colors of the visible spectrum are represented. In this manner, the visible wavelengths of light are substantially all present, resulting in a whiter appearance of the tooth.

A typical healthy tooth consists of an enamel surface, a dentin sub-layer beneath the enamel surface, and a central pulp chamber. The enamel surface comprises a highly calcified material that is preferably very hard and durable to allow chewing a wide variety of foods. The dentin sub-layer is also durable but considerably softer than the enamel. Dentin tends to have more of a yellowish hue compared to enamel, which tends to be white. Enamel may also be somewhat clear or translucent, which may enable yellowish tints of the dentin to show through the enamel. A tooth exhibiting a darker color than desired may also be the result of staining of the enamel, or even of the darker color of the pulp chamber being visible through the sometimes translucent enamel and dentin layers of the tooth.

Depending on the thickness, opacity and whiteness of the enamel, the color reflected by a person's teeth can vary widely between various shades of white, off-white, yellow, and yellow-green. The enamel surfaces of teeth can also accumulate plaque and tartar, which may increase their yellowish coloration.

In a preferred embodiment of the present invention, a small amount of dye is applied to a tooth, where that dye reflects a wavelength of light corresponding to violet or blue-violet. Because the violet or blue-violet reflected by the dye is a complementary color of the yellowish tints reflected by the tooth, the light reflected from the tooth with the dye has a whiter appearance than the tooth alone. In a preferred embodiment, a dye is used that has a dominant peak absorption wavelength within the range of 480 to 660 nm. Tarver, in the prior art, discloses the use of a dye or a combination of dyes that—though appearing violet or blue-violet—have multiple peak absorption wavelengths; using a dye with a dominant peak absorption wavelength can cause an otherwise yellowish tooth to appear substantially whiter than using a dye as disclosed in Tarver.

Referring now to FIG. 2, a combination of red and blue dyes that appear as violet may exhibit a spectral identity 2 having two peak absorption wavelengths, wherein a first peak 4 corresponds to a peak absorption wavelength unique to the red dye and a second peak 6 corresponds to a peak absorption wavelength unique to the blue dye. The resulting wavelength 2 is thus not truly violet, but simply the sum of the red wavelength 8 and the blue wavelength 10 as perceived by the eye. A dye according to the present invention exhibits a spectral identity 12 having a dominant peak absorption wavelength 14 corresponding to a point intermediate between the peak absorption wavelengths 4 and 6 for red and blue, respectively, and corresponding to the visible color of violet.

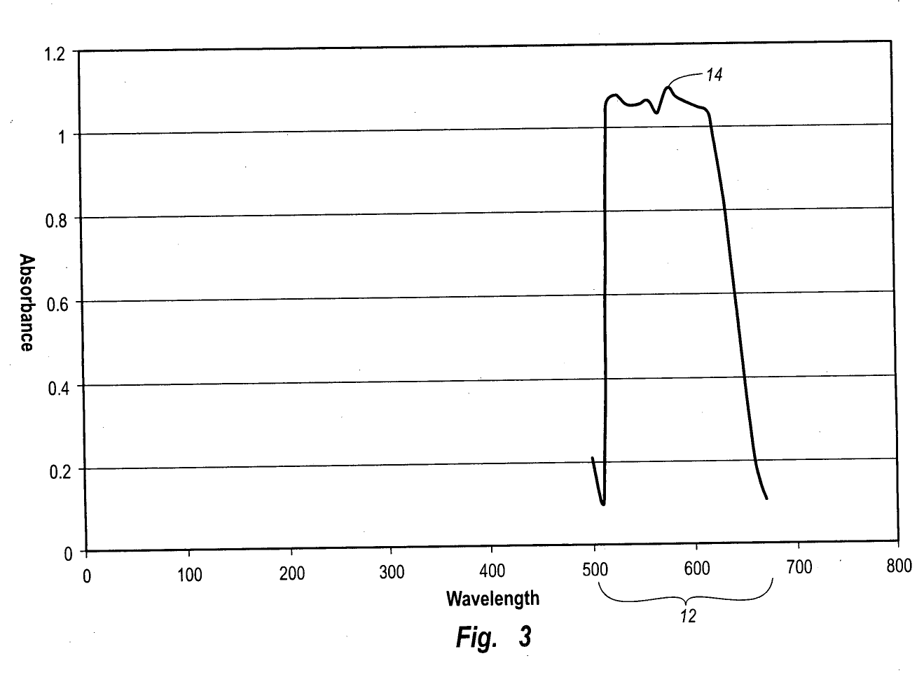

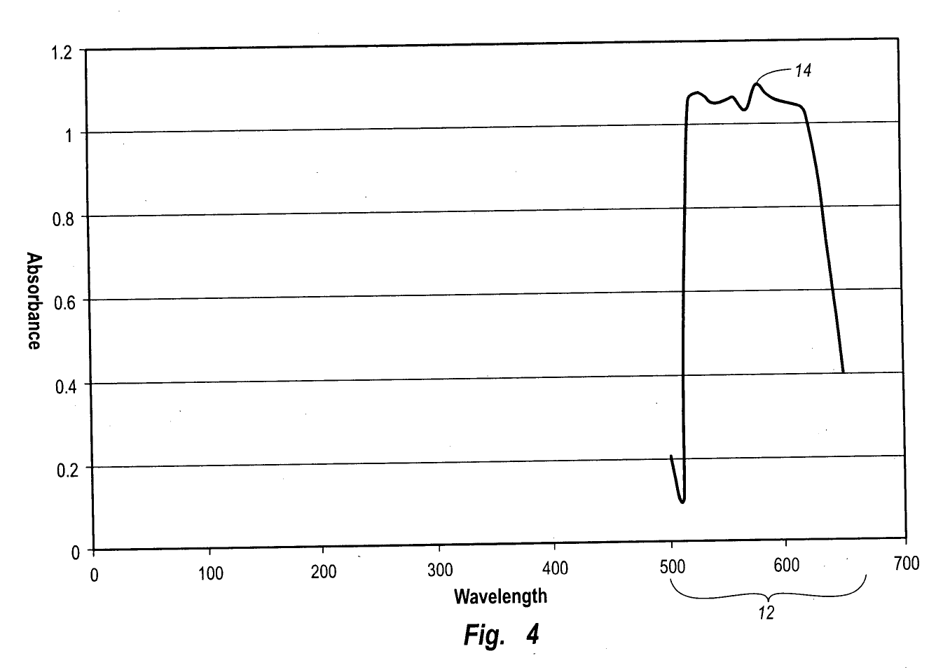

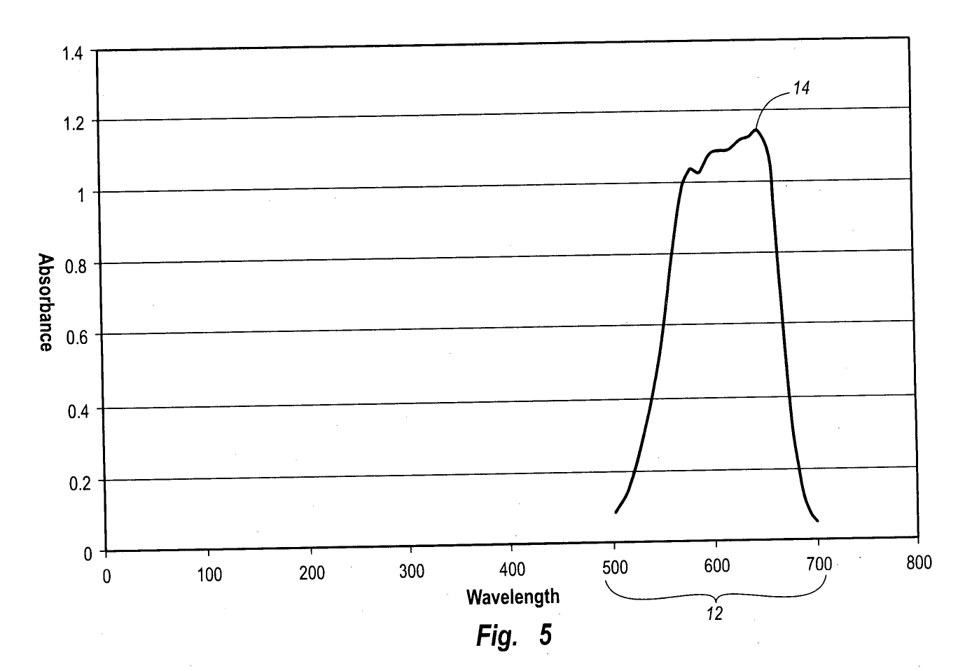

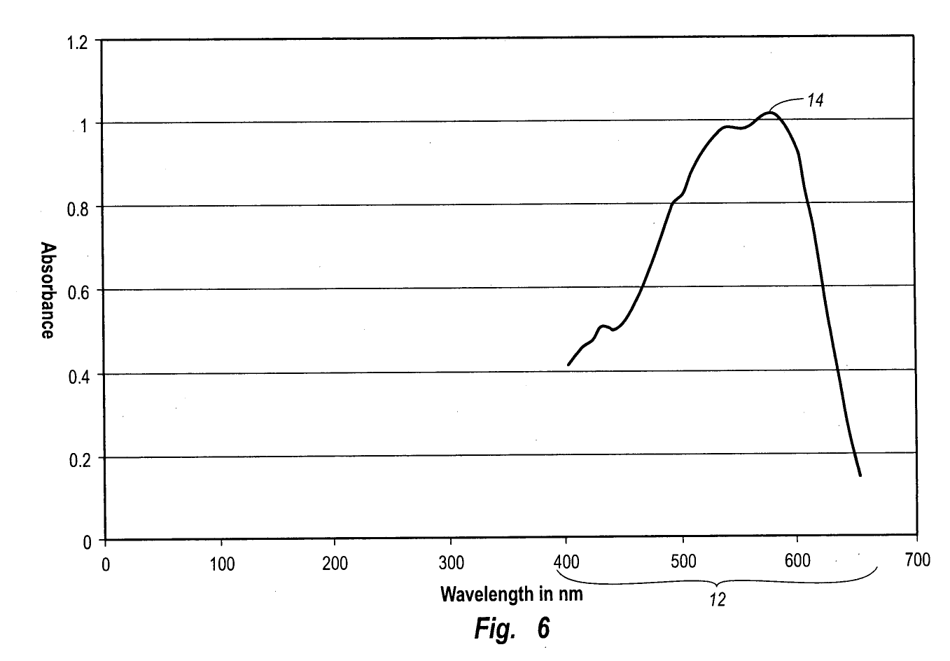

Referring now to FIGS. 3 through 6, a dye in accordance with a preferred embodiment of the present invention comprises a dye having a dominant peak absorption wavelength in the range of 480 to 660 nm, more preferably in a range from 500 to 620 μm, and most preferably in a range from 540 to 595 nm. A dye may comprise, for example, FD&C Red No. 3, D&C Red No. 33, FD&C Red No. 40, FD&C Blue No. 1, FD&C Blue No. 2, D&C Violet No. 2, Acid Violet No. 17, Carmine, Blueberry Additive, or any other artificial or natural colorant known to those in the art that, when used singly or in combination, has a dominant peak absorption wavelength in a range from 480 to 660 nm. The attached figures illustrate the spectral analysis of several dyes used according to preferred embodiments of the present invention, with the peak absorption wavelengths of each being shown. FIG. 3 illustrates a spectral analysis of Acid Violet No. 17; FIG. 4 illustrates a spectral analysis of D&C Red No. 33; FIG. 5 illustrates a spectral analysis of FD&C Blue No. 1; and FIG. 6 illustrates a spectral analysis of Carmine.

The distinction between a dye having one dominant peak absorption wavelength and a dye having multiple peak absorption wavelengths is of paramount importance to the effectiveness of a tooth dye as taught by the present invention. Indeed, results far superior to those achieved by dyes as disclosed by Tarver have been achieved by applying a dye having one dominant peak absorption wavelength, as disclosed herein.

Moreover, a dye according to a preferred embodiment of the present invention avoids the negative effects seen in the prior art, in which the red component of a dye was seen to cause staining of oral tissues. Red staining may occur where any dye made according to the prior art retains a discrete chemical structure corresponding to red dye. FD&C Red No. 3 is particularly prone to have a red staining effect due to exposed iodine molecules bordering the chemical structure.

In one embodiment of the present invention, the disclosed tooth whitening composition further comprises a carrier. A preferred class of carriers within the scope of the present invention comprises mouthwash or another hydrophilic carrier designed to wash or be sprayed onto a person's teeth. Such carriers enable a dye used according to the present invention to be evenly and uniformly dispersed over and absorbed into a person's teeth. Moreover, hydrophilic carriers enable rapid absorption of a dye by a person's teeth because such carriers are attracted by the aqueous environment surrounding teeth. By way of example and not limitation, a mouthwash or other hydrophilic carrier may comprise any combination of the following: water, alcohols, glycerin and other polyhydric alcohols, polyols, ketones, aldehydes, carboxylic acids, carboxylic acid salts, or amines.

According to another embodiment of the present invention, a gelling agent may be used as a carrier to yield a gel or more viscous liquid for application to a person's teeth. Such possible gelling agents include, but are not limited to, polycarboxylic acids, polycarboxylic acid salts, polysaccharides, polysaccharide derivatives, proteins, protein derivatives, polyalkylene oxides, fumed silica, and the like.

According to another embodiment of the present invention, a dye may be used in combination with a toothpaste or other dentifrice used to clean, protect, or whiten a person's teeth. For example, the present invention may be used in combination with brush-on applications, prophylaxis pastes for professional cleaning applications, temporary plastic strips that are topically applied to teeth, sealants, applications involving curing lights, applications involving laser whitening treatments, carbomide peroxide gel whitening treatments, peroxide gel whitening treatments, or any other tooth whitening, protecting, or cleaning treatment known to those in the art.

According to yet another embodiment of the present invention, a dye may be incorporated into a chewing gum that is used as a carrier, such that the dye is released gradually as the gum is chewed. A gum used as a carrier for a dye according to the present invention may comprise, for example, sorbitol, gum base, mannitol, glycerin, acesulfame potassium, aspartame, and flavoring.

In yet another embodiment of the present invention, the tooth whitening composition comprises a carrier such as a food product, and the disclosed dye. Preferably, the carrier in this embodiment comprises a starch, cereal, beverage, candy or confectionery. Food products that maximize contact time between the food product and a person's tooth, such as taffy, hard candy, beverages, and other liquid or slowly degradable food products are especially preferred as a means to facilitate absorption of the dye by a person's tooth.

In still another embodiment of the present invention, two dyes that when used together have the whitening effect disclosed herein may be administered in separate stages, rather than in a single operation. In one non-limiting example of this embodiment, a blue dye is administered within a mouthwash, followed by a red dye that is administered within a dentifrice. The cumulative effect of these operations may then yield the results disclosed herein.

A tooth whitening composition in accordance with the present invention may be quickly and easily implemented by, for example, administering the disclosed tooth whitening composition to a person for ordinary consumption where the composition comprises a carrier such as a food product, or by selectively applying the tooth whitening composition directly to a person's tooth by way of an applicator stick, a toothbrush, an adhesive, or any other means known to those in the art.

### EXAMPLE 1

A beverage within the scope of the present invention was prepared by mixing with purified water the following two components: FD&C Blue No. 1 in a concentration of 0.04 mg/ml and FD&C Red No. 3 in a concentration of 0.033 mg/ml.

Test subjects drinking this water experienced an increase in tooth whiteness, as measured by an X-rite ShadeVision™ tooth color measurement instrument.

### EXAMPLE 2

A food product within the scope of the present invention was prepared by mixing a carrier of 500 ml of Scope® brand mouthwash with a solution containing 3025 ul of blue solution and 3375 ul of red solution. The blue solution comprised FD&C Blue No. 1 in a concentration of 6.05 ul/ml, or 15 mg/ml in the final mixture. The red solution comprised FD&C Red No. 33 in a concentration of 6.75 ul/ml, or 15 mg/ml in the final mixture.

Test subjects used this mixture as a mouthwash and experienced an increase in tooth whiteness.

### EXAMPLE 3

A food product within the scope of the present invention was prepared by mixing a carrier of 50 g of Arm & Hammer® brand toothpaste with a solution containing 550 ul of red solution and 605 ul of blue solution. The blue solution comprised FD&C Blue No. 1 in a concentration of 12.10 ul/g, or 60 mg/ml in the final mixture. The red solution comprised FD&C Red No. 33 in a concentration of 11.00 ul/g, or 60 mg/ml in the final mixture.

Test subjects used this mixture as a dentifrice and experienced an increase in tooth whiteness.

### EXAMPLE 4

A food product within the scope of the present invention was prepared by adding Acid Violet No. 17 to a carrier of Scope® brand mouthwash in a concentration of 0.048 ml of dye to each ml of mouthwash.

Test subjects used this mixture as a mouthwash and experienced an increase in tooth whiteness.

What is claimed is:

1. A tooth whitening composition for whitening at least one tooth, wherein said tooth absorbs an initial wavelength of light, said initial wavelength of light corresponding to a first color, said composition comprising:
a dye having a dominant peak absorption wavelength, wherein said dominant peak absorption wavelength corresponds to a second color, and where said second color is a complementary color to said first color; and
a food product, wherein said food prodtict acts as a carrier for said dye.

2. The tooth whitening composition of claim 1, wherein said dominant peak absorption wavelength is in a range from 480 to 660 nm.

3. The tooth whitening composition of claim 1, wherein said dominant peak absorption wavelength is in a range from 480 to 500 nm.

4. The tooth whitening composition of claim 1, wherein said dominant peak absorption wavelength is in a range from 500 to 520 nm.

5. The tooth whitening composition of claim 1, wherein said dominant peak absorption wavelength is in a range from 520 to 540 nm.

6. The tooth whitening composition of claim 1, wherein said dominant peak absorption wavelength is in a range from 540 to 560 nm.

7. The tooth whitening composition of claim 1, wherein said dominant peak absorption wavelength is in a range from 560 to 580 nm.

8. The tooth whitening composition of claim 1, wherein said dominant peak absorption wavelength is in a range from 580 to 600 nm.

9. The tooth whitening composition of claim 1, wherein said dominant peak absorption wavelength is in a range from 600 to 620 nm.

10. The tooth whitening composition of claim 1, wherein said dominant peak absorption wavelength is in a range from 620 to 640 nm.

11. The tooth whitening composition of claim 1, wherein said dominant peak absorption wavelength is in a range from 640 to 660 nm.

12. The tooth whitening composition of claim 1, wherein said dye is selected from the group consisting of FD&C Red No. 3, D&C Red No. 33, FD&C Red No. 40, FD&C Blue No. 1, FD&C Blue No. 2, D&C Violet No. 2, Acid Violet No. 17, Carmine, and Blueberry Additive.

13. The tooth whitening composition of claim 1, wherein said food product is selected from the group consisting of a starch, a cereal, a beverage, a candy, and a confectionery.

14. A method for whitening teeth comprising:
providing a tooth whitening composition comprising a food product and a dye, said dye having a dominant peak absorption wavelength in a range from 480 to 660 nm; and
administering said tooth whitening composition to a person.

15. The method of claim 14, wherein said dominant peak absorption wavelength is in a range from 480 to 500 nm.

16. The method of claim 14, wherein said dominant peak absorption wavelength is in a range from 500 to 520 nm.

17. The method of claim 14, wherein said dominant peak absorption wavelength is in a range from 520 to 540 nm.

18. The method of claim 14, wherein said dominant peak absorption wavelength is in a range from 540 to 560 nm.

19. The method of claim 14, wherein said dominant peak absorption wavelength is in a range from 560 to 580 nm.

20. The method of claim 14, wherein said dominant peak absorption wavelength is in a range from 580 to 600 mm.

21. The method of claim 14, wherein said dominant peak absorption wavelength is in a range from 600 to 620 mm.

22. The method of claim 14, wherein said dominant peak absorption wavelength is in a range from 620 to 640 mm.

23. The method of claim 14, wherein said dominant peak absorption wavelength is in a range from 640 to 660 nm.
